(19) United States
(12) Patent Application Publication
(10) Pub. No.: US 2022/0160230 A1
(43) Pub. Date: May 26, 2022

(54) FUNDUS OCULI IMAGING DEVICE AND FUNDUS OCULI IMAGING METHOD USING SAME

(71) Applicant: ROOTEEHEALTH, INC., Seongnam-si, Gyeonggi-do (KR)

(72) Inventor: Kyung Min KOOK, Seongnam-si, Gyeonggi-do (KR)

(21) Appl. No.: 17/594,281

(22) PCT Filed: Apr. 11, 2019
(86) PCT No.: PCT/KR2019/004348

§ 371 Date: Oct. 8, 2021

(30) Foreign Application Priority Data
Apr. 10, 2019 (KR) 10-2019-0042192

(51) Int. Cl. A61B 3/12; A61B 3/00; A61B 3/15

(52) U.S. Cl. CPC A61B 3/12; A61B 3/152; A61B 3/0008

(57) ABSTRACT

This application relates to a fundus oculi imaging device and a fundus oculi imaging method including the same. In one aspect, the fundus oculi imaging device includes a housing and a first imaging module that is installed to be movable in the housing and captures a retinal image of an examinee. The fundus oculi imaging device may also include a light irradiation module moving along with the first imaging module in the housing and irradiating light to an eye of the examinee. The fundus oculi imaging device may further include a second imaging module installed on a side of the housing and capturing an image of a cornea or a pupil, to which light is irradiated from the light irradiation module, of the examinee.

### TECHNICAL FIELD

One or more embodiments of the disclosure relate to an apparatus and a method, and more particularly, to a fundus oculi imaging device and a fundus oculi imaging method.

### BACKGROUND ART

Fundus oculi refers to a posterior part of a retina in an eyeball, and through a fundus oculi examination, a central portion of the retina, e.g., a macula, an optic disc, and retinal blood vessels, etc. may be observed. In addition, severity of hypertension may be determined and diabetic complication related to eyes may be examined through the fundus oculi examination. A shape of the optic disc is used to diagnose various optic neuropathy such as glaucoma, increased intracranial pressure, optic neuritis, ischemic optic neuropathy, etc., and is essential for diagnosing retinal diseases such as macular degeneration, retinopathy of prematurity, etc. In particular, an early diagnosis of glaucoma and macular degeneration, which are two of three major causes of blindness, may be possible through the fundus oculi examination.

Because a fundus oculi camera according to the related art has to examine the fundus oculi of an examinee while being fixed to a certain position, the examinee has to visit medical facilities such as a hospital in order to get the fundus oculi examination, and it may be difficult to have the fundus oculi examination in an area lacking medical institutions.

In addition, the fundus oculi camera according to the related art irradiates light to an eyeball in order to improve optical performances. In order to capture a clear retinal image, a light source has to be evenly irradiated to the retina, and reflection of light from a cornea, a lens, a vitreous body, etc. has to be removed or minimized.

### DESCRIPTION OF EMBODIMENTS

### Technical Problem

The present disclosure provides a fundus oculi imaging device capable of aligning a position of an imaging module rapidly and precisely, and obtaining a clear and exact retinal image, and a fundus oculi imaging method using the fundus oculi imaging device.

### Technical Solution to Problem

According to an aspect of the present disclosure, a fundus oculi imaging device includes: a housing; a first imaging module that is installed to be movable in the housing and captures a retinal image of an examinee; a light irradiation module moving along with the first imaging module in the housing and irradiating light to an eye of the examinee; and a second imaging module installed on a side of the housing and capturing an image of a cornea or a pupil, to which light is irradiated from the light irradiation module, of the examinee.

### Advantageous Effects of Disclosure

A fundus oculi imaging device and method according to one or more embodiments of the present disclosure may obtain a clear and exact retinal image of an examinee. According to the fundus oculi imaging device and method, a position of a first imaging module is precisely aligned before capturing a retinal image, and thus, light from a light irradiation module may be exactly irradiated to an outline of a pupil such that a clear and bright retinal image may be obtained.

The fundus oculi imaging device and method according to one or more embodiments of the present disclosure may align the fundus oculi imaging device rapidly and accurately based on a corneal image. Information about a pupil and irradiated light may be extracted by using the corneal image obtained by a second imaging module, and a distance that needs to be adjusted is calculated by using the information to rapidly and accurately align a first imaging module.

According to the fundus oculi imaging device and method of the embodiments of the present disclosure, the fundus oculi imaging device is aligned a plurality of times, and thus, accuracy of the alignment is improved. In addition, even when a position of the fundus oculi imaging device is misaligned during an eye examination, the position may be corrected again. In detail, the alignment of the first imaging module in an x-axis direction and a y-axis direction is made respectively by using a retinal image and a corneal image, and thus, the alignment may be performed accurately. In addition, the alignment of the first imaging module in a z-axis direction may be performed by aligning the first imaging module in the z-axis direction within a range of forming the retinal image, and then aligning in the z-axis direction respectively by using the corneal image and the retinal image.

In the fundus oculi imaging device and method according to one or more embodiments of the present disclosure, the first imaging module is allowed to move in three-axial directions, and thus, the position may be accurately aligned. Because the first imaging module of the fundus oculi imaging device may be moved in the x-axis, y-axis, and z-axis directions, the position may be accurately aligned when a driving module receives a signal from a controller.

According to the fundus oculi imaging device and method of the embodiments of the present disclosure, the fundus oculi imaging device may be sensitively aligned. Because a second light source of a light irradiation module, which is used for the alignment, more sensitively affects the retinal image, the alignment of the first light source used to obtain the retinal image may be rapidly and accurately performed.

### BRIEF DESCRIPTION OF THE DRAWINGS

FIG. 1 is a diagram showing an example of a network environment according to an embodiment of the present disclosure.

FIG. 2 is a perspective view of a fundus oculi imaging device according to an embodiment of the present disclosure.

FIG. 3 is a perspective view showing an inside of the fundus oculi imaging device of FIG. 2.

FIG. 4 is a diagram schematically showing an optical structure of the fundus oculi imaging device of FIG. 2.

FIG. 5 is a diagram schematically illustrating the light irradiation module of FIG. 3.

FIG. 6 is an exploded perspective view showing a coupling relationship between some components of the fundus oculi imaging device of FIG. 2.

FIG. 7 is an exploded perspective view showing a coupling relationship between some components of the fundus oculi imaging device of FIG. 2.

FIG. 8 is a front view showing a front of the fundus oculi imaging device of FIG. 2.

FIG. 9 is a diagram showing an arrangement of a second imaging module of FIG. 8.

FIG. 10 is a front view showing another arrangement of the second imaging module of FIG. 8.

FIG. 11 is a block diagram showing a control relationship of the fundus imaging device of FIG. 2.

FIG. 12 is a block diagram of a first information extractor of FIG. 11.

FIG. 13 is a block diagram of a second information extractor of FIG. 11.

FIG. 14 is a flowchart illustrating a fundus oculi imaging method according to another embodiment of the present disclosure.

FIG. 15 is a flowchart illustrating a first information extraction method of FIG. 14.

FIG. 16 is a flowchart illustrating a second information extraction method of FIG. 14.

FIG. 17 is a flowchart illustrating a method of adjusting a position of a first imaging module of FIG. 14.

### BEST MODE

According to an aspect of the present disclosure, a fundus oculi imaging device includes: a housing; a first imaging module that is installed to be movable in the housing and captures a retinal image of an examinee; a light irradiation module moving along with the first imaging module in the housing and irradiating light to an eye of the examinee; and a second imaging module installed on a side of the housing and capturing an image of a cornea or a pupil, to which light is irradiated from the light irradiation module, of the examinee.

The fundus oculi imaging device may further include a controller obtaining first information about a pupil of the examinee or second information about irradiated light from the image captured by the second imaging module.

The apparatus may further include a driving module for moving the first imaging module, wherein the controller drives the driving module to align a position of the first imaging module based on the first information and the second information.

The controller may identify whether the light irradiated from the light irradiation module is reflected by the retina, from a retinal image of the examinee, the retinal image being captured by the first imaging module.

The controller may convert coordinates of the image captured by the second imaging module in order to make the converted image correspond to coordinates of the image captured by the first imaging module.

The fundus oculi imaging device may further include a shutter unit connected to the first imaging module and having a surface on which the second imaging module is installed.

The fundus oculi imaging device may further include an illumination unit spaced apart from the first imaging module and arranged at an edge of the shutter unit.

The second imaging module may be arranged to be inclined on a surface of the shutter unit to face a center of the pupil.

The light irradiation module may include: a pair of first light sources spaced apart from each other in a vertical direction based on a central axis of the first imaging module; and second light sources arranged next to the first light sources and closer to the central axis of the first imaging module than the first light sources.

According to another aspect of the present disclosure, provided is a fundus oculi imaging device including: a housing; a first imaging module that is installed to be movable in the housing and captures a retinal image of an examinee; a light irradiation module moving along with the first imaging module in the housing and irradiating light to an eye of the examinee; and a shutter unit closing one end of the housing, wherein the shutter unit includes: a shutter holder connected to the first imaging module such that the first imaging module is movable toward the examinee; and a shielding slider connected to the shutter holder and moving along a guide rail of the housing when the first imaging module moves in directions toward both eyes of the examinee.

The fundus oculi imaging device may further include a flexible shield member installed in front of the shutter holder and the shield slide, and closing a gap between the examinee and the housing.

The shield member may further include a slit provided in a recess portion, in which a nose of the examinee is inserted, and allowing a shape change of the recess portion.

According to another aspect of the present disclosure, provided is a fundus oculi imaging method including: irradiating light from a light irradiation module to an eye of an examinee, and moving a first imaging module to a region where a retina of the examinee is seen; capturing an image of a cornea or a pupil of the examinee by using a second imaging module; extracting first information about the pupil of the examinee from the image captured by the second imaging module; extracting second information about the light irradiated from the light irradiation module from the image captured by the second imaging module; and adjusting a position of the first imaging module based on the first information and the second information.

The adjusting of the position of the first imaging module may include: aligning a center of the pupil and an optical axis of the first imaging module to coincide with each other; aligning a pair of light irradiated from the light irradiation module to be symmetrical at a center of the pupil; and aligning the light irradiated from the light irradiation module, not to be reflected from the retina.

The fundus oculi imaging method may further include converting the image captured by the second imaging module, such that coordinates of the image captured by the second imaging module correspond to coordinates of the image captured by the first imaging module.

Other aspects, features and advantages of the disclosure will become better understood through the accompanying drawings, the claims and the detailed description.

### MODE OF DISCLOSURE

As the present disclosure allows for various changes and numerous embodiments, particular embodiments will be illustrated in the drawings and described in detail in the written description. The attached drawings for illustrating one or more embodiments are referred to in order to gain a sufficient understanding, the merits thereof, and the objectives accomplished by the implementation. However, the embodiments may have different forms and should not be construed as being limited to the descriptions set forth herein.

The embodiments will be described below in more detail with reference to the accompanying drawings. Those components that are the same or are in correspondence are rendered the same reference numeral regardless of the figure number, and redundant explanations are omitted.

While such terms as “first,” “second,” etc., may be used to describe various components, such components are not be limited to the above terms. The above terms are used only to distinguish one component from another.

An expression used in the singular encompasses the expression of the plural, unless it has a clearly different meaning in the context.

In the present specification, it is to be understood that the terms “including,” “having,” and “comprising” are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

It will be understood that when a layer, region, or component is referred to as being “formed on” another layer, region, or component, it may be directly or indirectly formed on the other layer, region, or component. That is, for example, intervening layers, regions, or components may be present.

Sizes of components in the drawings may be exaggerated for convenience of explanation. In other words, since sizes and thicknesses of components in the drawings are arbitrarily illustrated for convenience of explanation, the following embodiments are not limited thereto.

When a certain embodiment may be implemented differently, a specific process order may be performed differently from the described order. For example, two consecutively described processes may be performed substantially at the same time or performed in an order opposite to the described order.

In the embodiments below, when layers, areas, or elements or the like are referred to as being “connected,” it will be understood that they may be directly connected or an intervening portion may be present between layers, areas or elements. For example, when layers, areas, or elements or the like are referred to as being “electrically connected,” they may be directly electrically connected, or layers, areas or elements may be indirectly electrically connected and an intervening portion may be present.

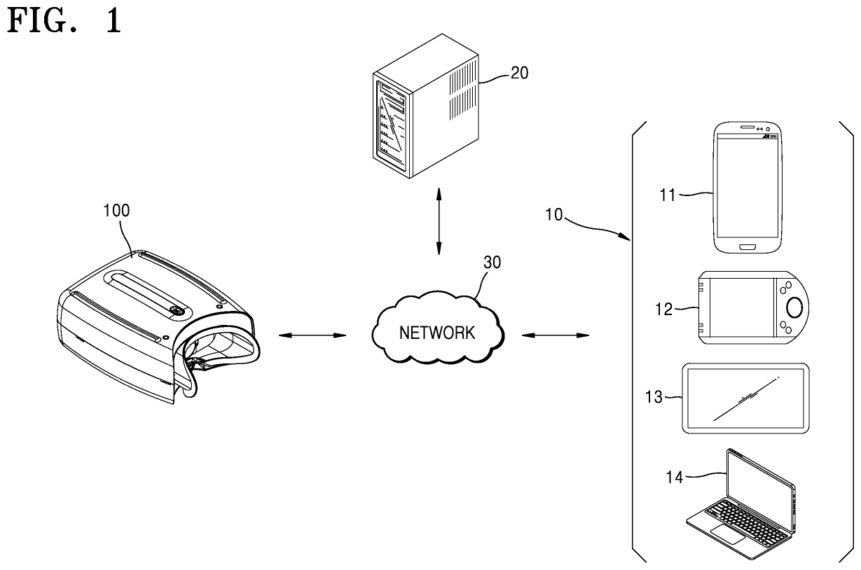

FIG. 1 is a diagram showing an example of a network environment according to an embodiment of the present disclosure.

FIG. 1 shows an example in which the network environment includes a user terminal 10, a server 20, a network 30, and a fundus oculi imaging device 100. FIG. 1 shows an example for describing the present disclosure, and the number of user terminals or the number of servers is not limited to the example shown in FIG. 1.

The user terminal 10 may be a fixed terminal implemented as a computer device or a mobile terminal. The user terminal 10 may include a terminal for transmitting data received from the fundus oculi imaging device 100 that will be described later, to the server 20. Also, the user terminal 10 may display data in the fundus oculi imaging device 100 described later, or may be a terminal manipulated by a third party. Examples of the user terminal 10 may include a smartphone, a mobile phone, a navigation system, a computer, a laptop computer, a digital broadcasting terminal, a personal digital assistant (PDA), a portable multimedia player (PMP), a tablet PC, etc. For example, a first user terminal 11 may communicate with second to fourth user terminals 12, 13, and 14 and/or the server 20 via the network 30 using a wired or wireless communication method.

The communication method is not particularly restricted, that is, the communication method may include a communication using a communication network (e.g., a mobile communication network, wired Internet, wireless Internet, broadcast network, etc.) that may be included in the network 30, and near distance wireless communication between devices. For example, the network 30 may include one or more arbitrary networks from among personal area network (PAN), local area network (LAN), campus area network (CAN), metropolitan area network (MAN), wide area network (WAN), broadband network (BBN), the Internet, etc. In addition, the network 30 may include, but is not limited to, one or more arbitrary networks from network topology including a bus network, a star network, a ring network, a mesh network, a star-bus network, a tree or hierarchical network, etc.

The server 20 may be implemented as a computer device or a plurality of computer devices communicating with the user terminal 10 via the network 30 to provide commands, codes, files, contents, services, etc.

For example, the server 20 may provide a file for installing an application to the first user terminal 11 connected through the network 30. In this case, the first user terminal 11 may install the application by using the file provided from the server 20. In addition, the first user terminal 11 may access the server 20 according to the control of an operating system (OS) and at least one program (e.g., a browser or an installed application) of the first user terminal 11, and may receive service or contents provided by the server 20. In another example, the server 20 may establish a communication session for data transmission/reception, and may route data transmission/reception between the user terminals 10 through the established communication session.

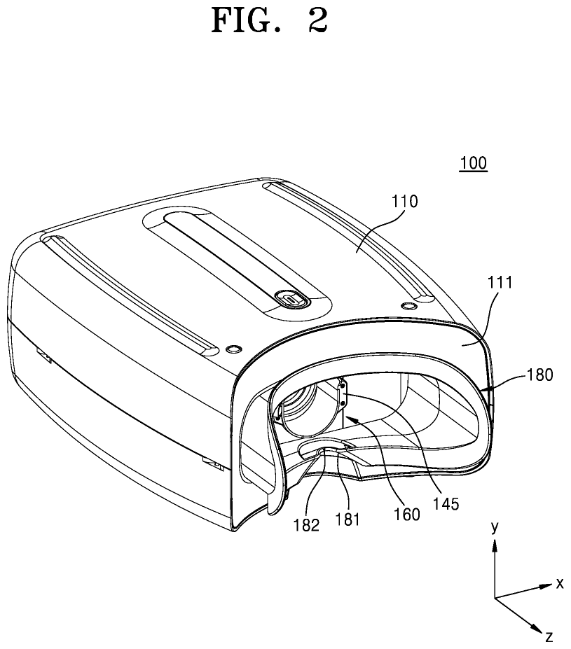

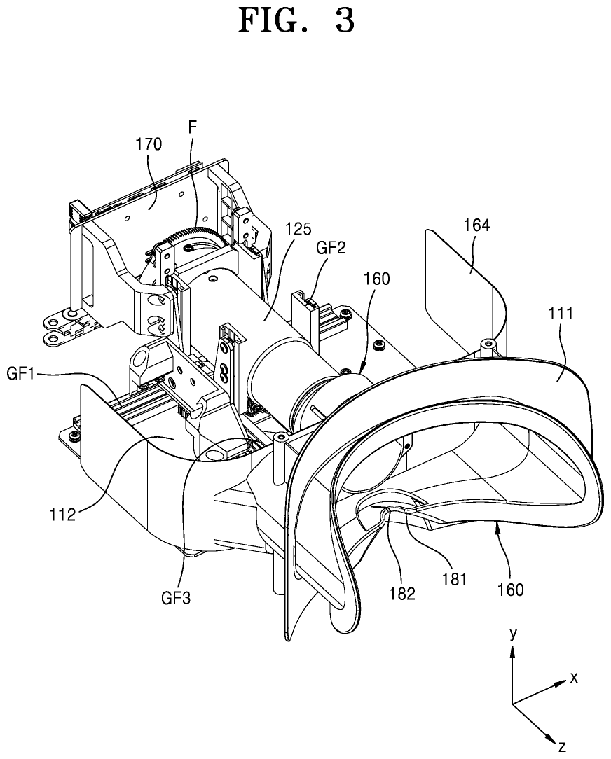

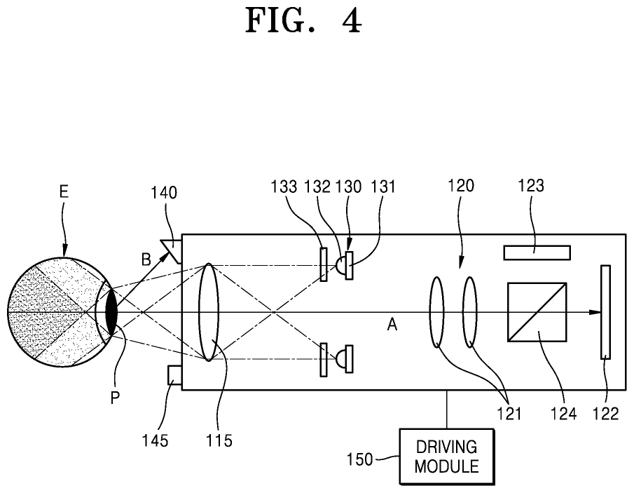

FIG. 2 is a perspective view of the fundus oculi imaging device 100 according to the embodiment of the present disclosure, FIG. 3 is a perspective view showing the inside of the fundus oculi imaging device 100 of FIG. 2, and FIG. 4 is a diagram schematically showing an optical structure of the fundus oculi imaging device 100 of FIG. 2.

Referring to FIGS. 2 to 4, the fundus oculi imaging device 100 according to the embodiment of the present disclosure may obtain an image of the fundus oculi, that is, a retina, while being worn by an examinee. The fundus oculi imaging device 100 may include a housing 110, a first imaging module 120, a light irradiation module 130, a second imaging module 140, an illumination unit 145, a driving module 150, a shutter unit 160, a controller 170, and a shield member 180.

Hereinafter, a first image axis A is defined as an axis from which the first imaging module 120 obtains an image, and a second image axis B is defined as an axis from which the second imaging module 140 obtains an image.

The housing 110 forms an outer appearance of the fundus oculi imaging device 100, and in the housing 110, components of the fundus oculi imaging device 100 may be arranged. A front end of the housing 110 has a curved shape such that a center thereof is recessed and a face of the examinee may be inserted.

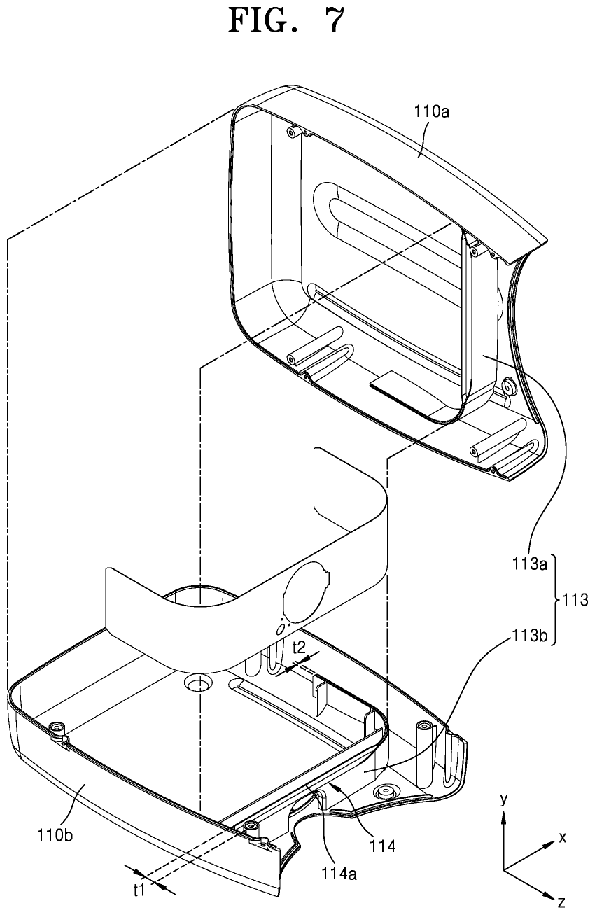

Referring to FIG. 7, the housing 110 may include a first case 110a covering an upper portion and a second case 110b covering a lower portion. In addition, a front cover 111 that is curved is disposed on the front end of the housing 110, and the shield member 180 may be installed on the front cover 111.

The first imaging module 120 may capture a retinal image of the examinee. The first imaging module 120 may capture the retinal image by using the light reflected from the retinas of the left eye, right eye, or both eyes of the examinee.

The first imaging module 120 is movably mounted in the inner space of the housing 110. The first imaging module 120 may include an optical system 121, an image sensor 122, a display unit 123, an optical path changing unit 124, and a barrel 125. Also, the first imaging module 120 may further include a polarizing plate (not shown) on the optical path to prevent cornea reflection and back-scattering.

Referring to FIG. 4, the optical system 121 is on a path of light A reflected from the retina, and may move for focusing. The optical system 121 may further include an auto focusing actuator allowing each lens to automatically focus.

The image sensor 122 may detect the light A reflected from the retina. The image sensor 122 may include a sensing unit (not shown) for detecting light of a specific wavelength band. For example, the image sensor 122 may include a complementary metal oxide semiconductor (CMOS) image sensor that captures an image when a light source of a visible light wavelength band and/or a light source of an infrared-ray wavelength band is used.

In an embodiment, the fundus oculi imaging device 100 according to the embodiment is arranged in the housing 110, and may further include the display unit 123 that provides a preset pattern to the left eye or the right eye of the examinee. The pattern image may denote an image including a gaze fixation point for fixing the eye of the examinee while photographing the retina. In another embodiment, the pattern image may include a pattern used for an eyesight test such as a colorblindness/color amblyopia test of the examinee.

The optical path changing unit 124 may further guide the pattern image provided from the display unit 123 to the retina of the examinee. The optical path changing unit 124 may change the path of the pattern image provided from the display unit 123 to guide the pattern image to the retina, and at the same time, may transmit the light reflected from the retina to guide the light to the image sensor 122.

The barrel 125 has the first imaging module 120 and the light irradiation module 130 arranged therein, and is connected to the driving module 150 to move in three-axial directions. When the barrel 125 moves in the three-axial directions for alignment, the first imaging module 120 and the light irradiation module 130 may move along with the barrel 125.

In detail, the fundus oculi imaging device 100 may include a guide frame for moving the barrel 125 along at least one of an x-axis, a y-axis, and a z-axis. The guide frame is supported by a base frame 112. When the controller 170 drives the driving module 150, the barrel 125 may move along the guide frame to align a spatial position thereof.

A first guide frame GF1 may move the barrel 125 in the x-axis direction. The barrel 125 may move along the first guide frame GF1 to move the first imaging module 120 to the left or right eye. A second guide frame GF2 may move the barrel 125 in the y-axis direction. The barrel 125 may move along the second guide frame GF2 to align a height of the first imaging module 120. A third guide frame GF3 may move the barrel 125 in the z-axis direction. The barrel 125 moves along the third guide frame GF3 so as to move the first imaging module 120 toward or away from the examinee, and the retinal image may be formed on the first imaging module 120.

In another embodiment, a focus adjusting end F for focusing may be arranged at a rear end of the barrel 125. The focus of the optical system 121 may be adjusted by rotating the focusing end F.

An objective lens 115 may be arranged in the barrel 125. The objective lens 115 may be disposed in front of the light irradiation module 130 and may guide the first light source L1 or the second light source L2 to an eyeball E.

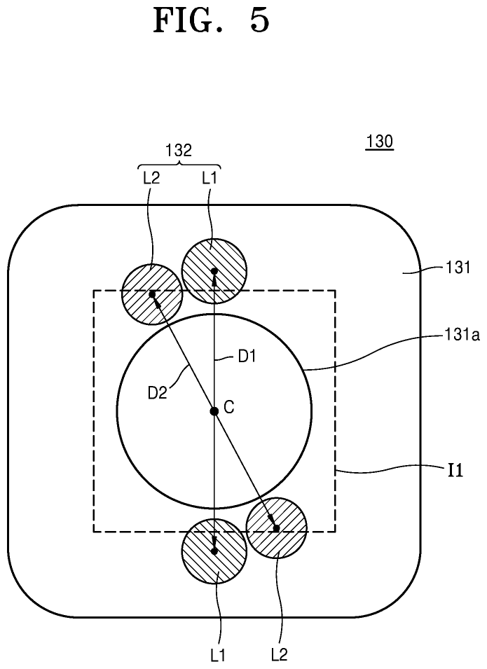

FIG. 5 is a diagram schematically showing the light irradiation module 130 of FIG. 3.

Referring to FIG. 5, the light irradiation module 130 moves along with the first imaging module 120 in the housing 110, and may irradiate light to the eyes of the examinee. The light irradiation module 130 is installed in the barrel 125, and a position of the barrel 125 may be adjusted to adjust the spatial position of the light irradiation module 130. The light irradiation module 130 may include a base 131, a light source unit 132, and a polarizing plate 133.

The base 131 includes an opening 131a formed in the center thereof, such that the first image axis A of the first imaging module 120 may pass through a point C.

The light source unit 132 may have a plurality of light sources. For example, the light source unit 132 may include the first light source L1 and the second light source L2.

A pair of the first light sources L1 is provided, and the first light sources L1 are spaced apart from each other in a vertical direction based on the central axis of the first imaging module 120. The first light sources L1 are arranged above and under the opening 131a. The first light sources L1 irradiate light to the eyeball E to obtain the retinal image. The first light sources L1 may have a visible ray wavelength band. In particular, the first light source L1 may emit white light. For example, the first light source L1 may emit white light of a wavelength band from 450 nm to 650 nm.

A pair of the second light sources L2 is provided, and is arranged next to the first light sources L1. The second light sources L2 are each arranged at a position spaced apart from each of the first light sources L1 by a certain angle. The second light sources L2 may have an infrared-ray wavelength band. The second light sources L2 emit light to the eyeball E to align the retinal image. That is, the second light sources L2 are used to align the first imaging module 120 before the first light sources L1 irradiate light. For example, the second light source L2 may emit infrared ray of a wavelength band from 750 nm to 950 nm.

In another embodiment, the first light sources L1 or the second light sources L2 may each include a plurality of light sources capable of irradiating light of a plurality of wavelength bands, and as necessary, may irradiate light by combining two or more from among the plurality of wavelength bands. For example, in order to identify a glucose level, the first light source L1 and/or the second light source L2 may irradiate light of a wavelength band from 650 nm to 750 nm and light of a wavelength band from 800 nm to 1300 nm. Alternatively, for autofluorescence imaging, the first light source L1 and/or the second light source L2 may irradiate light of a wavelength band from 470 nm to 490 nm, a wavelength band from 790 nm to 810 nm, and a wavelength of 450 nm. Here, the light of the wavelength band from 470 nm to 490 nm, the light of the wavelength band from 790 nm to 810 nm, and the light of the wavelength of 450 nm may be respectively used for autofluorescence imaging of lipofuscin, melanin, and flavoprotein. Alternately, in order to measure advanced glycation end products (AGEs), the first light source L1 and/or the second light source L2 may irradiate light of a wavelength band from 370 nm to 400 nm. Alternately, in order to measure oxygen saturation (hemoglobin, deoxyhemoglobin), the first light source L1 and/or the second light source L2 may irradiate light of a wavelength band from 570 nm to 580 nm, light of a wavelength of 750 nm, and light of a wavelength of 800 nm. The first light source L1 and/or the second light source L2 may irradiate light having different wavelength bands at once, or sequentially irradiate the light to the retina of the left eye or right eye of the examinee.

In addition, according to the arrangement of the first light source L1 and the second light source L2, the fundus oculi imaging device 100 may precisely align the first imaging module 120. A size of the retinal image captured by the first imaging module 120 may be referred to as I1 in FIG. 5. After aligning the position of the first imaging module 120 by using the second light source L2, the retinal image is obtained by using the first light source L1. It is important to align the position of the first imaging module 120 by irradiating the light from the second light source L2 before capturing the retinal image.

Because the first light source L1 is arranged in a height direction of the opening, the first light source L1 corresponds to an edge of the retinal image I1. On the other hand, because the second light source L2 is rotated from the first light source L1 by a preset angle, the second light source L2 is arranged around a corner of the retinal image I1. That is, the second light source L2 used to align the first imaging module 120 affects a wide area of the retinal image I1, and the first light source L1 used to obtain the retinal image affects a relatively narrow area of the retinal image I1.

The controller 170 aligns the first imaging module 120 after checking whether the light source is reflected from the retina through the retinal image I1. Because the second light source L2 affects a wide area, the light source reflected from the retina is displayed in the retinal image I1 more sensitively than the first light source L1. That is, when the position of the first imaging module 120 is adjusted by using whether the sensitive second light source L2 is reflected, the first light source L1 is not reflected and not displayed in the retinal image I1, and thus, an accurate retinal image may be obtained.

Also, the pair of the first light sources L1 is arranged to have a distance D1 therebetween and the pair of the second light sources L2 is arranged to have a distance D2 therebetween. The pair of second light sources L2 is arranged closer to the first image axis A of the first imaging module 120 than the pair of first light sources L1. That is, the second light source L2 is farther from the point C than the first light source L1.

Because the distance D1 between the second light sources L2 is shorter, the second light source L2 affects the large area of the retinal image I1, and because the distance D1 between the first light sources L1 is longer than the distance D2 between the second light sources L2, the first light sources L1 affects the small area of the retinal image I1. Therefore, when the position of the first imaging module 120 is adjusted by using whether the sensitive second light source L2 is reflected, the first light source L1 is spaced apart from the point C, and thus, the reflected light is not represented in the retinal image I1, and an accurate retinal image may be obtained.

The fundus oculi imaging device 100 according to the embodiment of the present disclosure may sensitively and accurately align the first imaging module 120 according to the arrangement of the first and second light sources L1 and L2 of the light irradiation module 130, and as such, the clear and accurate retinal image may be obtained.

The second imaging module 140 is installed at one side of the housing 110, and may capture an image of the cornea or a pupil, to which the light is irradiated from the light irradiation module 130, of the examinee. The second imaging module 140 may photograph the outside of the eyeball E to identify where in a pupil P the light irradiated from the light irradiation module 130 is located. The second imaging module 140 will be described in detail later.

The driving module 150 may move the first imaging module 120 in the internal space of the housing 110. Because the driving module 150 moves the barrel 125, an objective lens 115, the first imaging module 120, and the light irradiation module 130 arranged in the barrel 125 may be moved together.

The driving module 150 may adjust the position of the first imaging module 120 in at least three axes. The driving module 150 receives a signal from the controller 170 for moving the position of the barrel 125 along at least one of the x-axis, y-axis, and z-axis, and an actuator (not shown) is driven. When a distance for the barrel 125 to move is calculated by the controller 170 that will be described later, the driving module 150 may align the position of the barrel 125.

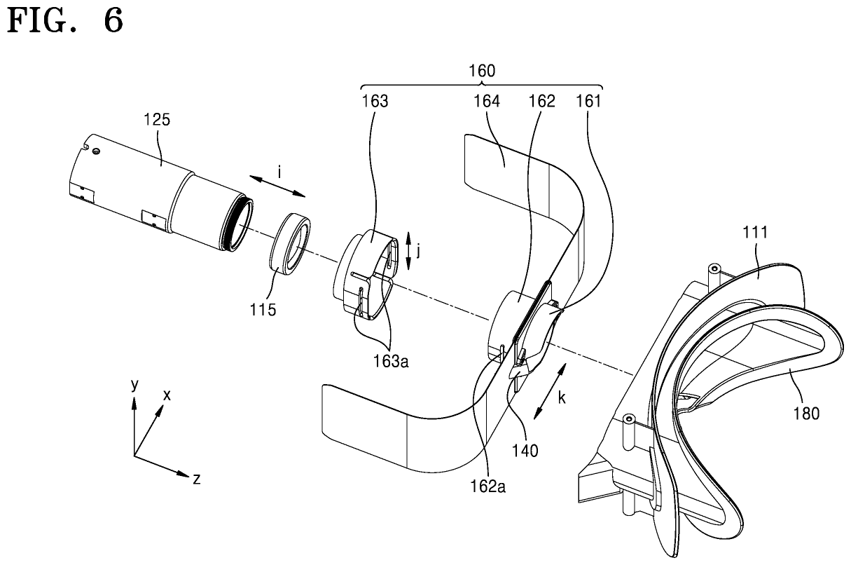

FIG. 6 is an exploded perspective view illustrating a coupling relationship of some components in the fundus oculi imaging device 100 of FIG. 2.

Referring to FIG. 6, the shutter unit 160 closes one end of the housing 110. The shutter unit 160 is installed in front of the housing 110, where the both eyes of the examinee are located, and may prevent external light from being incident in the fundus oculi imaging device 100.

The shutter unit 160 is connected to the first imaging module 120 by a shutter holder. The shutter holder may allow, for alignment, the first imaging module 120 to move toward the examinee. The shutter holder connects the first imaging module 120 and a shielding slider 164. The second imaging module 140 and the illumination unit 145 may be mounted outside the shutter holder. The shutter holder may include a first shutter holder 161, a second shutter holder 162, and a third shutter holder 163.

The first shutter holder 161 is arranged on the outer side of the shielding slider 164 and has an outer side surface thereof, on which the second imaging module 140 and the illumination unit 145 are installed. The second imaging module 140 and the illumination unit 145 may be installed at a location adjacent to an opening of the first shutter holder 161.

The second shutter holder 162 is arranged inside the shielding slider 164. The first shutter holder 161 and the second shutter holder 162 may be coupled to each other and then may be fixed to the shielding slider 164. An alignment protrusion 162a may be provided on one side of the second shutter holder 162. The alignment protrusion 162a is inserted into an alignment groove 163a of the third shutter holder 163 and allows the first imaging module 120 connected to the third shutter holder 163 to move in a direction j (y-axis).

The third shutter holder 163 is connected to the end of the barrel 125. The barrel 125 is inserted into the third shutter holder 163 and is allowed to move in a direction i (z-axis). That is, because the barrel 125 and the third shutter holder 163 are not fixed in the z-axis direction, when the driving module 150 moves the barrel 125 in the z-axis direction, the barrel 125 may be moved while maintaining the inserted state in the third shutter holder 163.

The alignment groove 163a may be formed in one side of the third shutter holder 163. The alignment protrusion 162a of the second shutter holder 162 is inserted into the alignment groove 163a, so as to allow the third shutter holder 163 to move in the direction j.

In another embodiment, an alignment groove may be formed in the second shutter holder 162, and an alignment protrusion may be formed on the third shutter holder 163.

FIG. 7 is an exploded perspective view illustrating a coupling relationship of some components in the fundus oculi imaging device 100 of FIG. 2.

Referring to FIG. 7, the shielding slider 164 closes the front end of the housing 110 to prevent external light from being incident in the fundus oculi imaging device 100. Also, because the shielding slider 164 may move to opposite sides, even when the first imaging module 120 is moved to the left eye or the right eye, the external light may be continuously prevented from being incident. The shielding slider 164 is connected to the first shutter holder 161 and the second shutter holder 162, and moves along with the first imaging module 120 when the first imaging module 120 moves in the directions to the both eyes (direction k) of the examinee.

The housing 110 includes a guide rail unit 113 for guiding the shielding slider 164 to move in the x-axis direction. A first guide rail 113a is installed in the first case 110a, and a second guide rail 113b is installed in the second case 110b. The first guide rail 113a and the second guide rail 113b are inserted in upper and lower ends of the shielding slider 164 and support the upper and lower ends of the shielding slider 164, but allow the shielding slider 164 to move in the direction k.

A front surface of the guide rail unit 113 has a first width t1 that is greater than a thickness of the shielding slider 164, and a side surface of the guide rail unit 113 has a second width t2 that is nearly the same as the thickness of the shielding slider 164. The shielding slider 164 inserted in the first width t1 of a wider end 114 may move within a predetermined range in the back-and-forth direction (z-axis). That is, even when the barrel 125 moves in the z-axis direction, the first shutter holder 161 may move forward within a predetermined range. The wider end 114 of the guide rail unit 113 may allow the shutter unit 160 to move in the z-axis direction.

The guide rail unit 113 has no mound on the front portion thereof, but a support wall 114a is installed behind the guide rail unit 113. Because the front of the guide rail unit 113 is opened, when the barrel 125 moves in the z-axis direction toward the eyeball E of the examinee, the shielding slider 164 may be allowed to move forward. That is, the front of the guide rail unit 113 may be opened to generate a degree of freedom for allowing the movement of the first imaging module 120. The support wall 114a may prevent the shielding slider 164 from entering the housing 110. Even when the barrel 125 is moved backward, the support wall 114a supports the shielding slider 164 to prevent the shielding slider 164 from moving backward.

Referring to FIGS. 6 and 7, the fundus oculi imaging device 100 allows the barrel 125 to move in the x-axis, y-axis, or z-axis direction, and thus, the first imaging module 120 may be accurately aligned. When the controller 170 drives the driving module 150 to move the barrel 125 in the direction k, the shielding slider 164 may also move along the guide rail unit 113. Also, when the barrel 125 moves in the direction j, the third shutter holder 163 may move in the y-axis direction with respect to the second shutter holder 162. Also, when the barrel 125 moves in the direction i, the barrel 125 moves while being inserted in the third shutter holder 163. Here, because there is no mound in front of the guide rail unit 113, the movement of the barrel 125 in the z-axis direction may be allowed.

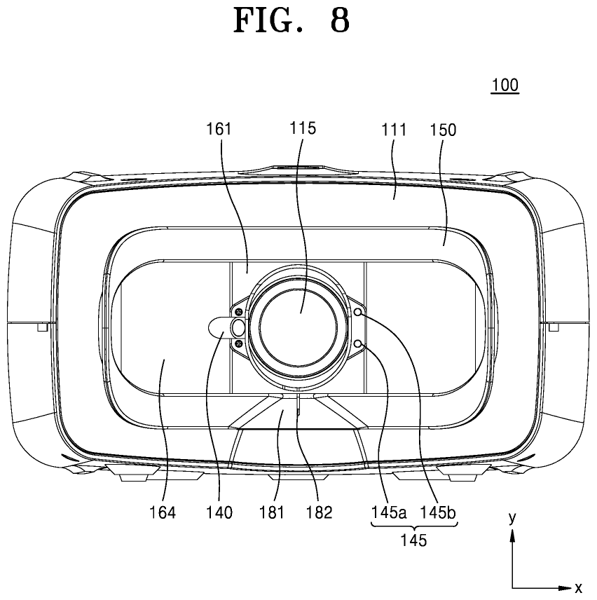

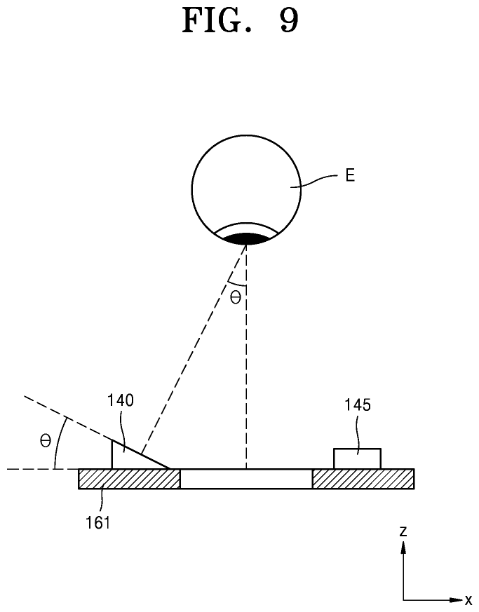

FIG. 8 is a front view of a front surface of the fundus oculi imaging device 100 of FIG. 2, and FIG. 9 is a diagram showing arrangement of the second imaging module 140 of FIG. 8.

Referring to FIGS. 8 and 9, the second imaging module 140 and the illumination unit 145 may be mounted on the shutter unit 160 to obtain a corneal image of the examinee.

The second imaging module 140 is arranged on one side of the first shutter holder 161 to capture an image of the outside of the eyeball E. When the light is irradiated from the light irradiation module 130, the second imaging module 140 may identify the light irradiated to the eyeball E through the captured corneal image. The controller 170 extracts data about a light source or a pupil based on the image obtained by the second imaging module 140, and aligns the first imaging module 120 based on the data.

The second imaging module 140 may be arranged to be inclined on a surface of the shutter unit 160 to face the center of the pupil P. That is, the second image axis B of the second imaging module 140 is different from the first image axis A, and is inclined with respect to the first image axis A. In FIG. 9, the second imaging module 140 is arranged to be inclined at an angle θ on the surface of the first shutter holder 161, and thus the image captured by the second imaging module 140 may face the center of the pupil P. The angle θ may be in a range of 15° to 60°.

The illumination unit 145 is spaced apart from the second imaging module 140 and is arranged at an edge of the shutter unit 160. The illumination unit 145 may illuminate such that the second imaging module 140 may obtain a clear image. In an embodiment, the illumination unit 145 and the second imaging module 140 may be arranged to face each other about the opening of the shutter unit 160.

The illumination unit 145 may have various wavelengths. The illumination unit 145 may have a light source that has an optimal wavelength according to the examinee. Here, the illumination unit 145 may have a plurality of light sources, or may adjust the wavelength by using a single light source.

Because pupil patterns vary depending on races, the illumination unit 145 may irradiate the wavelength that is optimized according to the race, and thus, the second imaging module 140 may obtain clear pupil pattern. For example, the illumination unit 145 may include a light source having a wavelength within a range of 600 nm to 1100 nm. In more detail, the illumination unit 145 may include a light source having a wavelength within a range of 850 nm to 1000 nm.

In another embodiment, the illumination unit 145 may include a plurality of light sources. For example, the illumination unit 145 may include a first illumination 145a and a second illumination 145b. The first illumination 145a and the second illumination 145b may have different brightnesses. For example, when the brightness of the first illumination 145a is less than the brightness of the second illumination 145b, a corneal image is obtained by using the first illumination 145a to reduce fatigue of the examinee. When the conical image is not clear, a corneal image may be obtained by using the second illumination 145b that is brighter.

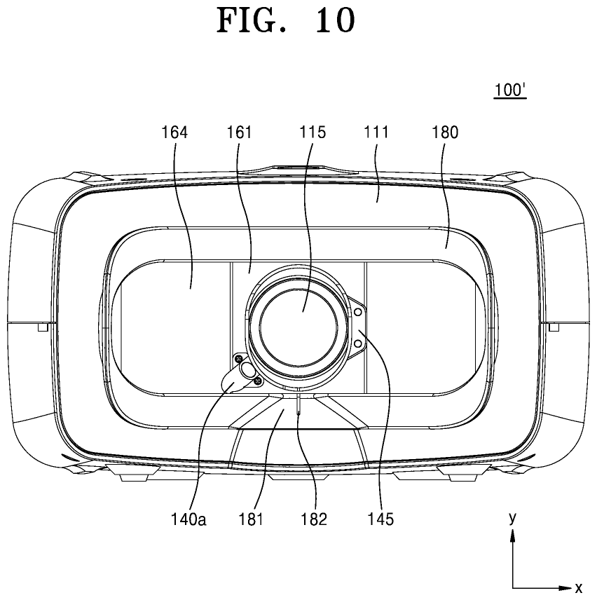

FIG. 10 is a front view showing another arrangement of the second imaging module 140 of FIG. 8.

Referring to FIG. 10, a second imaging module 140a may have a modified embodiment. The second imaging module 140a may be arranged at a lower portion in a diagonal direction of the opening in the shutter unit 160. The second imaging module 140a may secure the corneal image oriented from a lower direction of the eyeball E to upward, and thus, a clear image may be obtained.

In another embodiment, the second imaging module may include a plurality of camera modules. The number of camera modules is not limited to a specific number, and may be set according to an installation location, condition of the examinee, etc. For example, the second imaging module 140 of FIG. 8 and the second imaging module 140a of FIG. 10 may be both installed on the shutter unit 160.

Referring back to FIG. 3, the shield member 180 is installed in front of the shutter unit 160 and closes between the examinee and the housing 110. Because the shield member 180 is flexible, the face of the examinee may be in close contact with the shield member 180 so as to prevent the external light from being incident in the fundus oculi imaging device 100. The shield member 180 may maximize a darkroom effect by preventing the external light from being incident into the darkroom, such that the space between the examinee and the shielding slider may be provided as the darkroom.

The shield member 180 may have a recess portion 181 that is in contact with the nose of the examinee, and a slit 182 allowing a change in a shape of the recess portion 181. The slit 182 extends along a central line of the recess portion 181. Because the recess portion 181 in the shield member 180 has a thickness less than those of the other parts and the slit 182 is formed in the recess portion 181, it may be worn by examinees having various nose sizes.

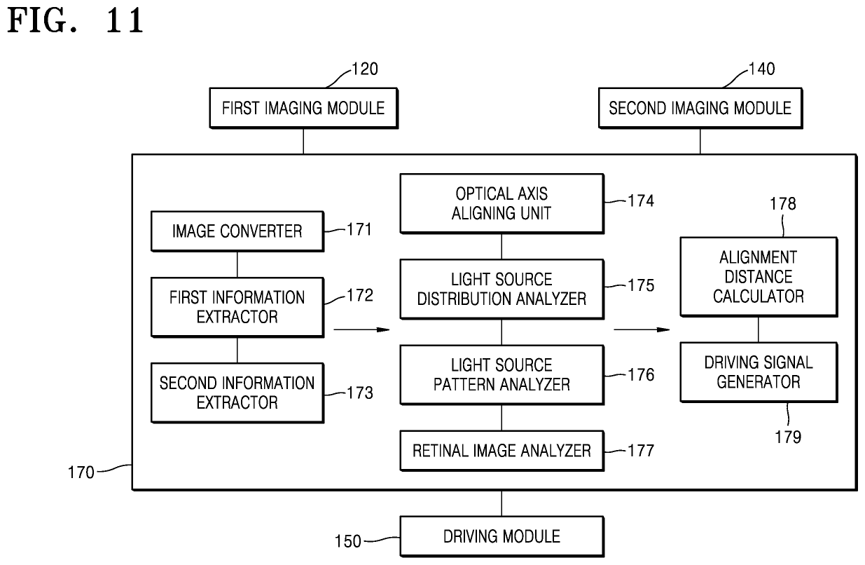

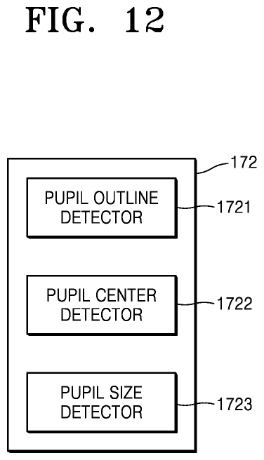

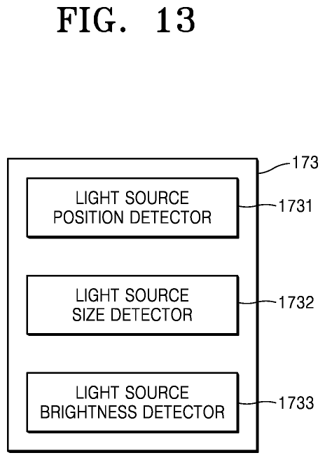

FIG. 11 is a block diagram illustrating a control relationship of the fundus imaging device 100 of FIG. 2, FIG. 12 is a block diagram of a first information extractor 172 of FIG. 11, and FIG. 13 is a block diagram of a second information extractor 173 of FIG. 11.

Referring to FIGS. 11 to 13, the controller 170 is connected to the first imaging module 120, the second imaging module 140, and the driving module 150. The controller 170 receives the retinal image from the first imaging module 120, receives the corneal image from the second imaging module 140, and may drive the driving module 150 to adjust the position of the first imaging module 120.

The controller 170 may obtain first information about the pupil of the examinee and second information about the light irradiated by the light irradiation module 130 from the image captured by the second imaging module 140. After that, the controller 170 may drive the driving module 150 to align the position of the first imaging module 120 based on the first information and the second information.

The controller 170 may include an image converter 171, the first information extractor 172, the second information extractor 173, an optical axis aligning unit 174, a light source distribution analyzer 175, a light source pattern analyzer 176, a retinal image analyzer 177, an alignment distance calculator 178, and a driving signal generator 179.

The image converter 171 may convert coordinates of the corneal image obtained by the second imaging module 140. The image converter 171 may convert the coordinates of the image captured by the second imaging module 140 to correspond to coordinates of the image captured by the first imaging module 120.

In another embodiment, the first information and the second information may be extracted by using the corneal image captured by the second imaging module 140 without image conversion.

Referring to FIG. 4, the first imaging module 120 obtains the retinal image incident in the first image axis A, but the second imaging module 140 obtains the corneal image incident in the second image axis B. Because a finally obtained image is a retinal image in the first image axis A, an operation for converting the corneal image to an image in the first image axis A. The image converter 171 may change the corneal image captured in the second image axis B into the first image axis A, such that the controller 170 may obtain the first and second information accurately and intuitively.

The first information extractor 172 may extract first information about the pupil of the examinee from the image captured by the second imaging module 140. The first information extractor 172 may obtain reference data by analyzing information about the pupil from the corneal image. The first information extractor 172 may include a pupil outline detector 1721, a pupil center detector 1722, and a pupil size detector 1723.

The pupil outline detector 1721 may detect an outline of the pupil from the capture corneal image. When the first light source L1 irradiates light along the outline of the pupil, the first imaging module 120 may secure a clear retinal image, and thus, it is necessary to define the outline of the pupil in the corneal image. The pupil outline detector 1721 may extract the outline of the pupil by using a difference in colors, a difference in color densities, etc.

The pupil center detector 1722 may detect a center of the pupil from the captured corneal image. In order to align the first imaging module 120 and the eyeball E, the center of the pupil has to coincide with the optical axis of the first imaging module 120. The pupil center detector 1722 may extract the center of the pupil based on information about the outline of the pupil.

The pupil size detector 1723 may detect a size of the pupil from the captured corneal image. Because a size and a shape of the pupil vary depending on each examinee, the size of the pupil needs to be exactly detected, and then, the light irradiation module 130 has to irradiate light to the outline of the pupil. The pupil size detector 1723 may detect the pupil size using a difference in color, a difference in color density, an area of the outline, etc.

The second information extractor 173 may extract second information about the light irradiated by the light irradiation module 130 from the image captured by the second imaging module 140. The second information extractor 173 may obtain reference data by analyzing information on light reflected from or transmitted through the corneal image. The second information extractor 173 may include a light source position detector 1731, a light source size detector 1732, and a light source brightness detector 1733.

The light source position detector 1731 may detect the position of the light source in the corneal image. Based on the data detected by the light source position detector 1731, the controller 170 may identify whether the pair of light sources is biased, symmetrical at the center of the pupil, and disposed inside the pupil.

The light source size detector 1732 may detect the size of the light source in the corneal image. A clear retinal image may be obtained only when a focus of the light source is placed on the outline of the pupil surface. The light source size detector 1732 may detect whether the focus of the light source is formed on the surface of the eyeball E by comparing the size of the light source.

The light source brightness detector 1733 may detect the brightness of the light source in the corneal image. The brightness of the light source relates to the focus of the light source. Therefore, it may be identified whether the focus of the light source is formed on the surface of the eyeball E by determining whether the brightness of the light source in the image falls within a preset range.

The optical axis aligning unit 174 aligns the optical axis, e.g., a central axis of the first imaging module 120. The optical axis aligning unit 174 may move the first imaging module 120 in the z-axis so that a retinal image is formed by the first imaging module 120. The retinal image may be generated or may not be generated in the first imaging module 120 according to a position of the optical system 121 in the z-axis direction. The optical axis aligning unit 174 may move the optical system 121 in the z-axis direction so that the retinal image is generated in the first imaging module 120.

The optical axis aligning unit 174 may align the first imaging module 120 in the x-axis and y-axis directions, so that the optical axis of the first imaging module 120 coincides with the center of the pupil. The first image axis A of the first imaging module 120 and the center of the pupil may be aligned with each other by moving the center of the retinal image captured by the first imaging module 120 in the x-axis and y-axis directions.

The light source distribution analyzer 175 may analyze distribution of the light irradiated from the light irradiation module 130 to analyze whether the light source irradiates the light correctly to the x-axis and y-axis positions. Based on the first information and the second information, it may be analyzed whether the pair of light sources is biased to a side from the center of the pupil, are arranged along the outline of the pupil, etc. Based on the data derived by the light source distribution analyzer 175, the first imaging module 120 may be moved and aligned in the x-axis or y-axis direction.

The light source pattern analyzer 176 may analyze whether the light source irradiates light accurately to the z-axis position by analyzing the pattern of light irradiated from the light irradiation module 130. The position of the light source in the z-axis direction may be aligned based on the first information and the second information, in particular, information about the size and shape of the pair of light sources represented in the corneal image. When the focus of the light source is located on the surface of the pupil, the light source has a brightness, a size, or a shape set in advance. The light source pattern analyzer 176 determines whether the brightness, size, or shape of the pair of light sources represented in the corneal image falls within a preset range, and aligns the first imaging module 120 in the z-axis direction based on the determination.

The retinal image analyzer 177 identifies whether the light irradiated from the light irradiation module 130 is reflected from the retina based on the retinal image of the examinee captured by the first imaging module, and then may align the first imaging module 120 in the z-axis direction. Even when the light source pattern analyzer 176 aligns the first imaging module 120 in the z-axis direction using the second light source L2, an error or a change may occur. The retinal image analyzer 177 may determine whether the first light source L1 or the second light source L2 is reflected from the final retinal image, and may align the first imaging module 120 in the z-axis direction.

The alignment distance calculator 178 calculates a distance that the first imaging module 120 has to move in the x-axis, y-axis, and z-axis directions based on the data derived by at least one of the optical axis aligning unit 174, the light source distribution analyzer 175, the light source pattern analyzer 176, and the retinal image analyzer 177.

The driving signal generator 179 is connected to the driving module 150 and drives the driving module 150 to align a spatial position of the first imaging module 120. The driving signal generator 179 generates a driving signal and transmits the generated driving signal to the driving module 150, and thus, the first imaging module 120 may move by the distance calculated by the alignment distance calculator 178.

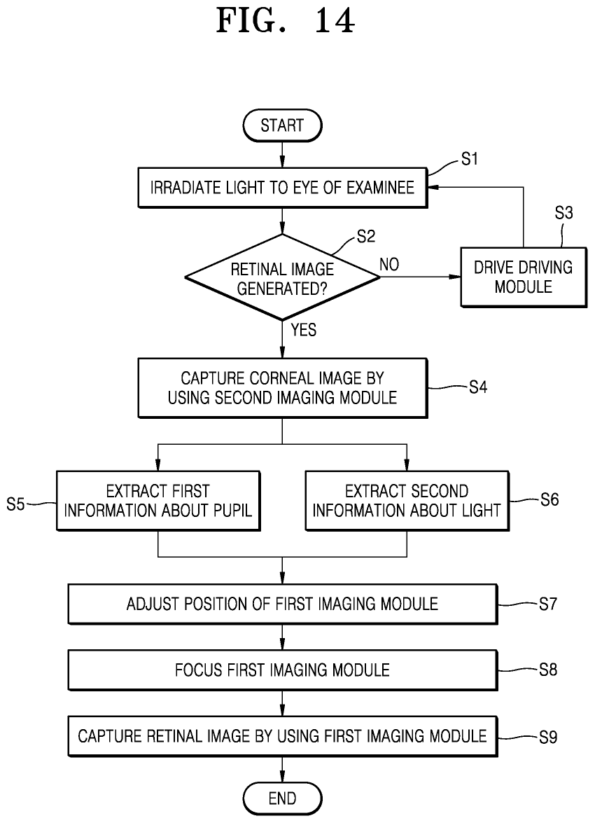

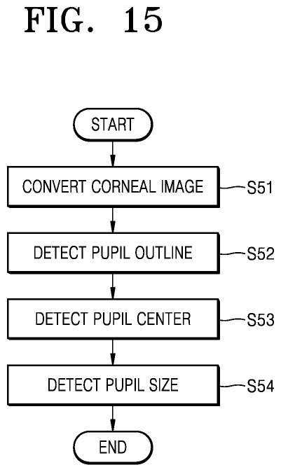

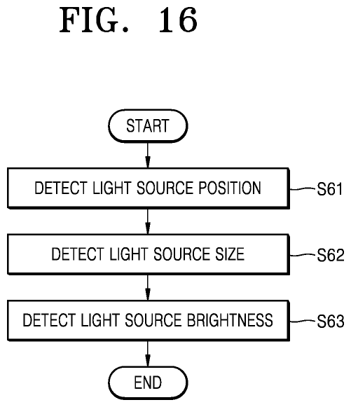

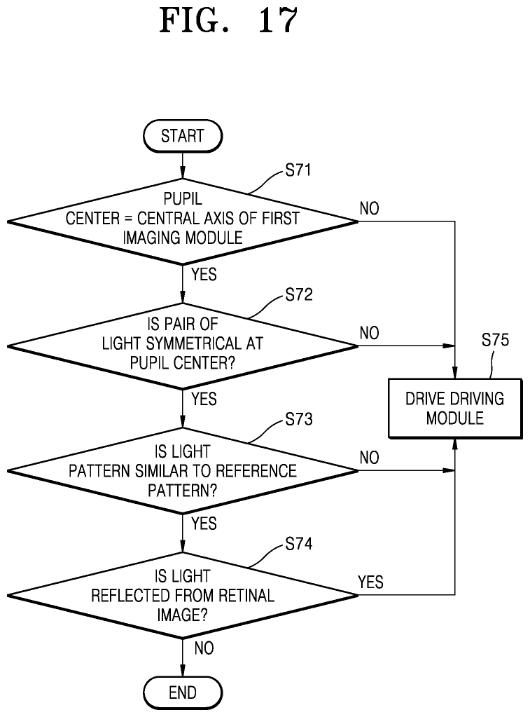

FIG. 14 is a flowchart illustrating a fundus oculi imaging method according to another embodiment of the present disclosure, FIG. 15 is a flowchart illustrating a first information extraction method of FIG. 14, FIG. 16 is a flowchart illustrating a second information extraction method of FIG. 14, and FIG. 17 is a flowchart illustrating a method of adjusting a position of a first imaging module of FIG. 14.

Referring to FIGS. 14 to 17, the fundus oculi imaging method includes: an operation of irradiating light from a light irradiation module to eyes of an examinee, and moving a first imaging module to a region where a retina of the examinee is seen; an operation of capturing an image of a cornea or a pupil of the examinee by using a second imaging module; an operation of extracting first information about the pupil of the examinee from an image captured by the second imaging module; an operation of extracting second information about the light irradiated from the light irradiation module from the image captured by the second imaging module; and an operation of adjusting a position of the first imaging module based on the first information and the second information.

In the operation of irradiating light to the eyes of the examinee from the light irradiation module and moving the first imaging module to the region where the retina of the examinee is seen, the first imaging module 120 is moved in the z-axis direction to a position where a retinal image is formed in the first imaging module 120. First, the light irradiation module 130 irradiates light to the eye of the examinee (S1), and determines whether the retinal image is generated (S2). When the retinal image is not formed in the first imaging module 120, the driving module 150 is driven to move the optical system 121 in the z-axis direction to the region where the retinal image is visible (S3).

In operation S4, in which the image of the cornea or the pupil of the examinee is captured by the second imaging module, the second imaging module 140 captures the corneal image while the light irradiation module 130 irradiates light to the eyeball E. The corneal image captured by the second imaging module 140 includes an image of the pupil and a light source.

In operation S5, in which the first information about the pupil of the examinee is extracted from the image captured by the second imaging module, pupil information of the examinee is secured based on the corneal image.

An operation of converting the corneal image (S51) may be further provided. As shown in FIG. 9, the image captured by the second imaging module 140 is an image captured from a side surface of the eyeball E, and does not correspond to the image captured by the first imaging module 120. Therefore, an operation of converting the image captured by the second imaging module may be performed by the image converter 171, such that coordinates of the image captured by the second imaging module 140 may correspond to coordinates of the image captured by the first imaging module 120.

The pupil outline detector 1721 detects the outline of the pupil based on the corneal image (S52), the pupil center detector 1722 detects the pupil center based on the corneal image (S53), and the pupil size detector 1723 detects the pupil size based on the corneal image (S54). The pupil outline, pupil center, and pupil size are used as reference data for aligning the first imaging module 120.

In operation S6, in which second information about the light irradiated from the light irradiation module is extracted from the image captured by the second imaging module, the information about irradiated light is secured based on the corneal image.

The light source position detector 1731 detects the light source position in the corneal image (S61), the light source size detector 1732 detects the light source size in the corneal image (S62), and the light source brightness detector 1733 detects the brightness of the light source in the corneal image (S63).

In an operation of adjusting the position of the first imaging module based on the first information and the second information (S7), a distance to be moved by the first imaging module 120 for alignment is calculated based on the first information and the second information, and the driving module 150 is driven to move the first imaging module 120. In operation S7, in which the position of the first imaging module is adjusted, 1) it is determined whether the pupil center and the optical axis of the first imaging module coincide (S71), 2) it is determined whether the pair of light irradiated from the light irradiation module are symmetrical at the pupil center (S72), 3) it is determined whether a pattern of the pair of light irradiated from the light irradiation module is similar to a reference pattern (S73), and 4) it is determined whether the light irradiated from the light irradiation module is reflected from the retina (S74). Here, the driving module 150 is driven to move the first imaging module 120 for alignment (S75).

In detail, in operation S71 in which it is determined whether the pupil center and the optical axis of the first imaging module coincide, the optical axis aligning unit 174 may align the first imaging module 120 in the x-axis and y-axis directions such that the optical axis of the first imaging module 120 may coincide with the pupil center. The barrel 125 may be moved in the x-axis and y-axis directions such that the first image axis A, e.g., the optical axis of the first imaging module 120, may coincide with the pupil center.

In FIGS. 14 and 17, operation S71 is shown to be executed after analyzing the corneal image in the second imaging module 140, but one or more embodiments are not limited thereto, that is, operation S71 may be performed before capturing the corneal image. In another embodiment, the optical axis aligning unit 174 may determine whether the pupil center coincides with the optical axis of the first imaging module 120 before or after operation S2, and based on the determination, the first imaging module 120 may be moved in the x-axis and y-axis directions.

In operation S72 in which it is determined whether the pair of light irradiated from the light irradiation module is symmetrical at the pupil center, the light source distribution analyzer 175 may analyze the distribution of light irradiated from the light irradiation module 130 to analyze whether the light source is irradiated accurately to the x-axis and y-axis positions. Based on the first information and the second information, it may be analyzed whether the pair of light sources is biased to a side from the center of the pupil, are arranged along the outline of the pupil, etc. Based on the data derived from the light source distribution analyzer 175, the alignment distance calculator 178 calculates a distance to be moved in the x-axis and y-axis directions, and the driving signal generator 179 drives the driving module 150 to move and align the first imaging module 120.

In operation S73, in which it is determined whether the pattern of the pair of light irradiated from the light irradiation module is similar to the reference pattern, the light source pattern analyzer 176 may analyze the pattern of light irradiated from the light irradiation module 130 to identify whether the first imaging module 120 is appropriately positioned along the z-axis direction. It may be analyzed where the focus of the light source is positioned in the z-axis direction based on the first information and the second information, in particular, information about the sizes and shape of the pair of light sources represented in the corneal image. When the focus of the light source is located on the surface of the pupil, the light source has a brightness, size, or shape set in advance. The light source pattern analyzer 176 may analyze whether the focus of the light source is properly formed by determining whether the brightness, size, or shape of the pair of light sources represented in the corneal image falls within a preset range. The alignment distance calculator 178 calculates the distance to be moved in the z-axis direction based on the data derived by the light source pattern analyzer 176 and the driving signal generator 179 may drive the driving module 150 to move and align the first imaging module 120 in the z-axis direction.

In operation S74, in which it is determined whether the light irradiated from the light irradiation module is reflected from the retina, it is determined whether there is reflective light in the retinal image generated by the first imaging module 120. In operations S72 and S73, the position of the first imaging module 120 is adjusted based on the corneal image, and in operation S74, the position of the first imaging module 120 is adjusted again based on the retinal image.

In detail, the retinal image analyzer 177 identifies whether the light irradiated from the light irradiation module 130 is reflected from the retina based on the retinal image of the examinee captured by the first imaging module 120, and then may align the first imaging module 120 in the z-axis direction. Even when the light source pattern analyzer 176 aligns the first imaging module 120 in the z-axis direction using the second light source L2, an error or a change may occur. The retinal image analyzer 177 may determine whether the first light source L1 or the second light source L2 is reflected from the final retinal image, and may align the first imaging module 120 in the z-axis direction.

Operation S75, in which the driving module 150 is driven to move the first imaging module 120 for alignment, may be performed after executing each of operations S71 to S74. For example, operations S71 and S75 are executed until operation S71 is finished, and then, operation S72 is performed. In addition, operations S72 and S75 may be performed until operation S72 is finished.

In another embodiment, operation S75 may be performed after performing at least one of operations S71 to S74. A plurality of operations selected from among operations S71 to S74 may be simultaneously performed, and as a result, operation S75 is performed. After finishing the selected operations, remaining operations and operation S75 may be performed.

In another embodiment, when operation S75 is performed, a certain operation may be repeatedly performed after returning to the certain operation. For example, when operation S75 is performed after operation S73, the process may return to a set start, and then, operations may be performed from operation S71, or operations may be performed again from operation S72 set in advance.

Operation S75, in which the first imaging module 120 is moved by driving the driving module 150, may be selectively performed a plurality of times in operation S7, in which the position of the first imaging module is adjusted based on the first information and the second information. In operation S75, a feedback control function for aligning the first imaging module 120 may be provided, and thus, the position of the first imaging module 120 may be accurately set.

In operation S8, in which the first imaging module is focused, the focus of the optical system 121 may be adjusted. After aligning the spatial position of the first imaging module 120, the focus of the first imaging module 120 is adjusted.

In operation S9, in which the retinal image is captured by the first imaging module, the first light source L1 of the light irradiation module 130 irradiates light, and the first imaging module 120 obtains the retinal image. Because the spatial position of the first imaging module 120 is aligned in operation S7, the first light source L1 is arranged on the outline of the pupil. That is, because the first light source L1 irradiates light to the accurate position, the first light source L1 may obtain clear retinal image.

A fundus oculi imaging device and method according to the present disclosure may obtain a clear and exact retinal image of an examinee. According to the fundus oculi imaging device and method, a position of the first imaging module 120 is precisely aligned before capturing a retinal image, and thus, light from the light irradiation module 130 may be exactly irradiated to an outline of a pupil such that a clear and bright retinal image may be obtained.

The fundus oculi imaging device and method according to the present disclosure may align the fundus oculi imaging device rapidly and accurately based on a corneal image. Information about a pupil and irradiated light may be extracted by using the corneal image obtained by the second imaging module 140, and a distance that needs to be adjusted is calculated by using the information to rapidly and accurately align the first imaging module 120.

According to the fundus oculi imaging device and method of the present disclosure, the fundus oculi imaging device is aligned a plurality of times, and thus, accuracy of the alignment is improved. In addition, even when a position of the fundus oculi imaging device is misaligned during an eye examination, the position may be corrected again. In detail, the first imaging module 120 in the x-axis and y-axis directions may be precisely aligned because the alignment is performed by using the retinal image in operation S71 and using the corneal image in operation S72. In addition, the first imaging module 120 is aligned in the z-axis direction within a range in which the retinal image is formed in operation S2, and is aligned in the z-axis direction by using the corneal image in operation S73. In addition, the first imaging module 120 may be aligned in the z-axis direction by using the retinal image in operation S74.

In the fundus oculi imaging device and method according to the present disclosure, the first imaging module 120 is allowed to move in three-axial directions, and thus, the position thereof may be accurately aligned. In the fundus oculi imaging device 100, the first imaging module 120 may be moved in the x-axis, y-axis, and z-axis directions by the shutter unit 160, and thus, when the driving module 150 receives a signal from the controller 170, the position may be accurately aligned.

According to the fundus oculi imaging device and method of the present disclosure, the fundus oculi imaging device may be sensitively aligned. Because a second light source of the light irradiation module 130, used for the alignment, more sensitively affects the retinal image, the alignment of the first light source used to obtain the retinal image may be rapidly and accurately performed.

While the disclosure has been particularly shown and described with reference to exemplary embodiments thereof, it will be understood by those of ordinary skill in the art that various changes in form and details may be made therein without departing from the spirit and scope as defined by the following claims. Therefore, the scope sought to be protected of the disclosure shall be defined by the appended claims.

### INDUSTRIAL APPLICABILITY

The present disclosure provides a fundus imaging device and a fundus oculi imaging method. In addition, the embodiments of the present disclosure may be applied to industrial cases where a retina, a fundus oculus, an eyeball, a cornea, etc. are to be photographed.

1. A fundus oculi imaging device comprising:
a housing;
a first imaging module installed to be movable in the housing and configured to capture a retinal image of an examinee;
a light irradiation module configured to move along with the first imaging module in the housing and irradiate light to an eye of the examinee; and
a second imaging module installed on a side of the housing and configured to capture an image of a cornea or a pupil, to which light is irradiated from the light irradiation module, of the examinee.

2. The fundus oculi imaging device of claim 1, further comprising a controller configured to obtain first information about a pupil of the examinee and second information about irradiated light from the image captured by the second imaging module.

3. The fundus oculi imaging device of claim 2, further comprising:
a driving module configured to move the first imaging module,
wherein the controller is configured to drive the driving module to align a position of the first imaging module based on the first information and the second information.

4. The fundus oculi imaging device of claim 2, wherein the controller is configured to identify whether the light irradiated from the light irradiation module is reflected by the retina, from a retinal image of the examinee, the retinal image being captured by the first imaging module.

5. The fundus oculi imaging device of claim 2, wherein the controller is configured to convert coordinates of the image captured by the second imaging module in order to make the converted image correspond to coordinates of the image captured by the first imaging module.

6. The fundus oculi imaging device of claim 1, further comprising a shutter unit connected to the first imaging module and having a surface on which the second imaging module is installed.

7. The fundus oculi imaging device of claim 6, further comprising an illumination unit spaced apart from the first imaging module and arranged at an edge of the shutter unit.

8. The fundus oculi imaging device of claim 6, wherein the second imaging module is arranged to be inclined on a surface of the shutter unit to face a center of the pupil.

9. The fundus oculi imaging device of claim 1, wherein the light irradiation module includes:
a pair of first light sources spaced apart from each other in a vertical direction based on a central axis of the first imaging module; and
second light sources arranged next to the first light sources and closer to the central axis of the first imaging module than the first light sources.

10. A fundus oculi imaging device comprising:
a housing;
a first imaging module installed to be movable in the housing and configured to capture a retinal image of an examinee;
a light irradiation module configured to move along with the first imaging module in the housing and irradiate light to an eye of the examinee; and
a shutter unit configured to close one end of the housing,
wherein the shutter unit includes:
a shutter holder connected to the first imaging module such that the first imaging module is movable toward the examinee; and
a shielding slider connected to the shutter holder and configured to move along a guide rail of the housing when the first imaging module moves in directions toward both eyes of the examinee.

11. The fundus oculi imaging device of claim 10, further comprising a flexible shield member installed in front of the shutter holder and the shielding slider, and configured to close a gap between the examinee and the housing.

12. The fundus oculi imaging device of claim 11, wherein the shield member includes a slit provided in a recess portion, in which a nose of the examinee is inserted, and configured to allow a shape change of the recess portion.

13. A fundus oculi imaging method comprising:
irradiating light from a light irradiation module to an eye of an examinee, and moving a first imaging module to a region where a retina of the examinee is seen;
capturing an image of a cornea or a pupil of the examinee by using a second imaging module;
extracting first information about the pupil of the examinee from the image captured by the second imaging module;
extracting second information about the light irradiated from the light irradiation module from the image captured by the second imaging module; and
adjusting a position of the first imaging module based on the first information and the second information.

14. The fundus oculi imaging method of claim 13, wherein the adjusting of the position of the first imaging module includes:
aligning a center of the pupil and an optical axis of the first imaging module to coincide with each other;
aligning a pair of light irradiated from the light irradiation module to be symmetrical at a center of the pupil; and
aligning the light irradiated from the light irradiation module, not to be reflected from the retina.

15. The fundus oculi imaging method of claim 13, further comprising converting the image captured by the second imaging module, such that coordinates of the image captured by the second imaging module correspond to coordinates of the image captured by the first imaging module.
